(19) United States
(12) Patent Application Publication
(10) Pub. No.: US 2008/0039920 A1
(43) Pub. Date: Feb. 14, 2008

(54) Tethered Self-Expanding Stent Delivery System

(75) Inventors: James C. Peacock, San Carlos, CA (US); Michael J. Lee, Santa Rosa, CA (US)

Correspondence Address: KIRKPATRICK & LOCKHART PRESTON GATES ELLIS LLP;ATTN: Daniel S. Kim, 1900 MAIN STREET, SUITE 600, IRVINE, CA, 92614-7319, US

(73) Assignee: MEDLOGICS DEVICE CORPORATION, Santa Rosa, CA (US)

(21) Appl. No.: 11/463,291

(22) Filed: Aug. 8, 2006

(51) Int. Cl. A61F 2/06

(52) U.S. Cl. 623/1.11

(57) ABSTRACT

The present invention relates to devices, systems and methods to maintain self-expanding stents in a compressed form during delivery to and positioning at a treatment site. The devices, systems and methods of the present invention can provide for more accurate stent positioning and can improve the deliverability of self-expanding stents. Designs of the devices, systems and methods of the present invention include slotted designs, tethered designs and encapsulating flexible sheath designs.

### FIELD OF THE INVENTION

The present invention relates to devices, systems and methods to maintain self-expanding stents in a compressed form during delivery to a treatment site. The devices, systems and methods of the present invention can provide for more accurate stent positioning and can improve the deliverability of self-expanding stents.

### BACKGROUND OF THE INVENTION

Cardiovascular disease, including atherosclerosis, is the leading cause of death in the United States. The medical community has developed a number of methods and systems for treating coronary disease, some of which are specifically designed to treat the complications resulting from atherosclerosis and other forms of coronary artery narrowing.

An important development for treating atherosclerosis and other forms of vascular narrowing is percutaneous transluminal angioplasty, and, in the specific instance of coronary artery disease, percutaneous transluminal coronary angioplasty, hereinafter collectively referred to as “angioplasty.” The objective of angioplasty is to enlarge the lumen (inner tubular space) of an affected vessel by radial hydraulic expansion. The procedure is accomplished by inflating a balloon within the narrowed lumen of the affected vessel. Radial expansion of the affected vessel occurs in several different dimensions, and is related to the nature of the plaque narrowing the lumen. Soft, fatty plaque deposits are flattened by the balloon, while hardened deposits are cracked and split to enlarge the lumen. The wall of the vessel itself is also stretched when the balloon is inflated.

Unfortunately, while the affected vessel can be enlarged thus improving blood flow, in some instances the vessel recloses chronically (“restenosis”), closes down acutely (“abrupt reclosure”) or reoccludes (all hereinafter referred to as “reclosure”), negating the positive effect of the angioplasty procedure. Such reclosure frequently necessitates repeat angioplasty or open heart surgery. While such reclosure does not occur in the majority of cases, it occurs frequently enough that such complications comprise a significant percentage of the overall failures of the angioplasty procedure, for example, twenty-five to thirty-five percent of such failures.

To lessen the risk of reclosure, various devices have been proposed for mechanically keeping the affected vessel open after completion of the angioplasty procedure. Such endoprostheses (generally referred to as “stents”), are typically inserted into the vessel, positioned across the lesion or stenosis, and then expanded to keep the passageway clear. The stent provides a scaffold which overcomes the natural tendency of the vessel walls of some patients to reclose, thus maintaining the patency of the vessel and resulting blood flow.

Stents typically fall into two general categories: (1) those that are expandable upon application of a controlled force (often through the inflation of a balloon portion of a dilatation catheter); and (2) those that are self-expanding.

Self-expanding stents generally are made from materials that exhibit superelastic properties above a particular transitional temperature. That is to say, the material expands rapidly once it is heated above its transitional temperature. For example, most currently available self-expanding stents are made from nitinol which has a transitional temperature around about 20° C. Thus, a nitinol stent can be compressed and maintained in the compressed form without restraint so long as the stent remains at a temperature of less than about 20° C. When a nitinol stent reaches a temperature around about 20° C. or higher, however, the stent will begin to rapidly expand in a radial direction. Self-expanding stents can also be formed from materials such as, without limitation, Elgiloy® with a platinum core.

During the delivery of such self-expanding stents within a vessel to be treated, the stent reaches its transitional temperature almost as soon as it is introduced into the body, i.e. well before it is actually released into the vessel and comes into contact with the bloodstream. Accordingly, the stent must be maintained in a compressed form within a delivery catheter until it is positioned at its intended treatment site. When the stent is released into the vessel, because it has already reached its transitional temperature, it will expand almost instantaneously to its maximum radial diameter.

During delivery to a treatment site, most compressed self-expanding stents are contained within a closed space or chamber at the distal end of a delivery catheter system, the chamber being defined between an inner core member and an outer retractable sheath. The retractable sheath functions to maintain the stent in the compressed form until the stent is positioned at its intended treatment site. Once the stent is positioned in its intended treatment site within a vessel, the outer sheath can be retracted in the direction proximal to the distal end of the delivery catheter, thus exposing and releasing the self-expanding stent. As the outer retractable sheath is withdrawn, sections of the self-expanding stent expand as they are exposed. Once this outer sheath is completely retracted, the self-expanding stent has been fully deployed into the vessel. Delivery systems of this type are described in, for example, U.S. Pat. Nos. 6,391,050; 6,375,676; and 5,77,669.

A problem with the described stent delivery catheters and systems is that as the outer retractable sheath is withdrawn, the self-expanding stent is gradually exposed in a relatively slow unidirectional manner from the distal end of the self-expanding stent towards its proximal end. This effect of outer retractable sheaths causes a number of clinical problems during deployment of a self-expanding stent. For example, when the distal end of a self-expanding stent is exposed and allowed to expand before the rest of the stent, the distal end of the stent may contact the vessel wall before the rest of the stent is deployed, resulting in an “anchoring edge.” Once such an “anchoring edge” is created, the stent should no longer be moved or repositioned because any further movement can cause irritation and trauma to the vessel wall (caused by the metallic stent edges moving against the delicate inner surface of the vessel wall). Therefore, when using most currently-available stent designs, once the distal end of the stent is fully expanded, the stent is essentially locked into a position and further repositioning is problematic.

The inability to reposition a stent with such an “anchoring edge” is compounded by another problem created by outer retractable sheaths, i.e. the “spring effect.” The “spring effect” occurs when the withdrawal of an outer retractable sheath causes a self-expanding stent to behave like a spring. Specifically, as an outer retractable sheath is removed, the stored energy in the compressed stent is suddenly released. This sudden release can cause the stent to move in a distal direction in an unpredictable way, an effect that can be exaggerated in self-expanding stents having a short overall length.

Another difficulty that can be encountered during the release of self-expanding stents when using conventional delivery systems is related to a “push-pull” phenomenon. The “push-pull” phenomenon is encountered most dramatically in long catheter delivery systems, which are placed in tortuous vessels. The “push-pull” phenomenon presents itself as forward motion of the inner core of a delivery catheter in relation to the retractable sheath at the distal end of the system as the sheath is being retracted to expose the stent. In practical terms, the “push-pull” effect can result in the forward motion of the self-expanding stent thereby also resulting in inaccurate positioning.

Finally, the outer retractable sheaths associated with commonly-used delivery catheters are relatively stiff in nature, and as noted, hold the self-expanding stent in a compressed form from the outside of the self-expanding stent. These features can negatively affect the deliverability of self-expanding stents in at least two ways. First, the stiff nature of outer retractable sheaths provides less flexible delivery catheters that are more difficult to navigate through curved vessels. Second, the presence of an outer retractable sheath around the outside of a compressed self-expanding stent increases the diameter of the delivery catheter. This increase also can negatively affect the deliverability of the stent. Thus, a need for devices, systems and methods that address the identified problems would be beneficial. The present invention provides such devices, systems and methods.

### SUMMARY OF THE INVENTION

The devices, systems and methods of the present invention address the above-identified problems associated with commonly-used self-expanding stent delivery systems. First, the devices, systems and methods of the present invention can minimize stent movement during deployment, thus helping to enable more accurate positioning of the stent at the vessel treatment site. Second, the methods and systems of the present invention can reduce the “spring effect” and resulting “jump” of the stent caused by the spring effect. Third, the methods and systems of the present invention can minimize the “push-pull” phenomenon associated with commonly-used self-expanding stent delivery systems. Finally, the devices, systems and methods of the present invention can improve the deliverability of self-expanding stents by providing more flexible delivery catheter ends with smaller diameters.

The devices, systems and methods of the present invention achieve these benefits by providing devices and self-expanding stent delivery catheters that do not rely on outer retractable sheaths to allow expansion of a compressed self-expanding stent. Embodiments according to the present invention achieve this benefit by utilizing longitudinal or circumferential slot designs, longitudinal or circumferential tether designs or encapsulating flexible sheath designs.

The devices, systems and methods adopting slot designs of the present invention utilize a device comprising a cylindrically-shaped material with a plurality of slots wherein the slots are spaced on the material to accept sections of a self-expanding stent wherein the material has a tensile strength that is sufficient to maintain the self-expanding stent in a compressed form when the accepted sections of the stent are positioned within the slots. Slots on devices according to the present invention can be, without limitation, longitudinal and/or circumferential on the device.

Embodiments utilizing slotted designs of the present invention can include a stent delivery system comprising a delivery catheter, a device as described above and a self-expanding stent wherein the self-expanding stent is held in a compressed form by the device and wherein the device as a whole or individual segments thereof can be retracted, rotated or expanded by one or more controlling mechanisms of the delivery catheter such that the portions of the self-expanding stent positioned within the slots are released from the slots when the portion of the device containing the slot is retracted, rotated or expanded. In some embodiments according to the present invention, the whole device is retracted, rotated or expanded substantially simultaneously by the one or more controlling mechanisms such that the device releases the length of the self-expanding stent substantially simultaneously. In other embodiments according to the present invention, segments of the device are retracted, rotated or expanded sequentially by the one or more controlling mechanisms such that the device releases sections of the self-expanding stent sequentially.

Devices, systems and methods adopting tethering designs comprise a catheter and one or more tethers wherein the one or more tethers can maintain a self-expanding stent in its compressed form when the self-expanding stent is disposed over the distal end of the catheter. Certain embodiments of these devices, systems and methods further comprise a self-expanding stent positioned over the distal end of the catheter that is maintained in a compressed form by the one or more tethers.

Tethers according to the present invention can be loosened or broken through a method selected from the group consisting of inflating a ballon; actuating a control wire; applying current; applying heat; applying electrical charge; and combinations thereof. Tethers can be loosened or broken substantially simultaneously, individually or in groups. When loosened or broken, tethers not attached to the catheter can, without limitation, dissolve, degrade or be collected into a receptacle for removal from the treatment site. Alternatively, catheters according to the present invention can comprise a substrate to which the one or more tethers are attached. The substrate can be, without limitation, an inflatable balloon or a sleeve thereover.

Tethers can comprise a material or form selected from the group consisting of polyamides, polyolefins, polyesters, polyimides, polyurethanes, metals, wires, ribbons, and combinations thereof. In certain embodiments, these materials can be perforated.

In devices, systems, and methods adopting encapsulating flexible sheath designs of the present invention, the sheath provides channels that accept portions of a self-expanding stent to restrain it from expansion. Expansion of the encapsulating flexible sheath causes the channels to expel the portions of the self-expanding stent within them, allowing the stent to expand substantially simultaneously.

One encapsulating flexible sheath design according to the present invention includes a device comprising a flexible sheath and one or more partially open channels wherein the partially open channels accept portions of a stent within them and can retain the stent in a compressed form during delivery to a treatment site. The partially open channels can extend continuously along the length of the device or can extend intermittently along the length of the device.

The described device can be manufactured to be placed over a balloon catheter for the deployment of the stent. When the balloon portion of the balloon catheter is expanded, the partially open channels open further releasing portions of the stent held therein. Further, after the stent is released from the partially open channels, the device can be used to ensure deployment of the stent by rotating and expanding the device.

In another embodiment, the stent can comprise one or more bioactive materials.

### BRIEF DESCRIPTION OF THE FIGURES

FIGS. 1A-1D depict various non-limiting exemplary stent configurations that can be used as stent body portions in accordance with the present invention.

FIGS. 2A-2D depict appropriate longitudinal slotted designs for the stent configurations depicted in FIGS. 1A-1D.

FIGS. 3A-3D depict the stent configurations depicted in FIGS. 1A-1D positioned within the longitudinal slotted designs depicted in FIGS. 2A-2D.

FIGS. 4A-4C depict a stent appropriate for use with a circumferential slotted design according to the present invention (4A), an appropriate tubular slotted device (4B) and the stent of 4A restrained in the device of 4B (4C).

FIGS. 5A-5C depict an alternative stent appropriate for use with an circumferential slotted design according to the present invention (5A), an alternative appropriate tubular slotted device (5B) and the stent of 5A restrained in the device of 5B (5C).

FIGS. 6A-6B depicted longitudinal tethering approaches according to the present invention.

FIGS. 7A-7B depict one embodiment of a circumferential tethering approach according to the present invention (7A) and balloon expansion that can release these tethers (7B).

FIGS. 8A-8D depict additional embodiments of circumferential tethering (FIGS. 8A-8C) and a close up view depicting one method of providing the tethers of the present invention (8D).

FIGS. 9A and 9B depict an additional alternative embodiment of tethering according to the present invention.

FIGS. 10A-10C depict an end view of an encapsulating flexible sheath with channels according to the present invention with a stent held compressed within the sheath (10A), the stent released after expansion of the sheath (10B) and the stent expanded further by expansion of the sheath (10C).

FIGS. 11A-11C depict an oblique view of an encapsulating flexible sheath of the present invention with channels along its whole length (11A); an oblique view of an encapsulating flexible sheath with channels along portions of its length (11B) and the encapsulating flexible sheath depicted in 11B over an expansion balloon (11C).

### DEFINITION OF TERMS

Bioactive Materials: As used herein the phrase, “bioactive materials” refers to any organic, inorganic, or living agent that is biologically active or relevant. For example, a bioactive material can be a protein, a polypeptide, a polysaccharide (e.g. heparin), an oligosaccharide, a mono- or disaccharide, an organic compound, an organometallic compound, or an inorganic compound. It can include a living or senescent cell, bacterium, virus, or part thereof. It can include a biologically active molecule such as a hormone, a growth factor, a growth factor producing virus, a growth factor inhibitor, a growth factor receptor, an anti-inflammatory agent, an antimetabolite, an integrin blocker, or a complete or partial functional insense or antisense gene. It can also include a man-made particle or material, which carries a biologically relevant or active material. An example is a nanoparticle comprising a core with a drug and a coating on the core.

Bioactive materials also can include drugs such as chemical or biological compounds that can have a therapeutic effect on a biological organism. Bioactive materials include those that are especially useful for long-term therapy such as hormonal treatment. Examples include drugs for contraception and hormone replacement therapy, and for the treatment of diseases such as osteoporosis, cancer, epilepsy, Parkinson's disease and pain. Suitable biological materials can include, e.g., anti-inflammatory agents, anti-infective agents (e.g., antibiotics and antiviral agents), analgesics and analgesic combinations, antiasthmatic agents, anticonvulsants, antidepressants, antidiabetic agents, antineoplastics, anticancer agents, antipsychotics, and agents used for cardiovascular diseases such as anti-restenosis and anti-coagulant compounds. Exemplary drugs include, but are not limited to, antiproliferatives such as paclitaxel and rampamycin, everolimus, tacrolimus, des-aspartate angiotensin I, exochelins, nitric oxide, apocynin, gamma-tocopheryl, pleiotrophin, estradiol, heparin, aspirin and 5-hydroxy-3-methylglutaryl-coenzyme A reductase (HMG-CoA) reductase inhibitors such as atorvastatin, cerivastatin, fluvastatin, lovastatin, pravastatin, rosuvastatin, simvastatin, etc.

Bioactive materials also can include precursor materials that exhibit the relevant biological activity after being metabolized, broken-down (e.g. cleaving molecular components), or otherwise processed and modified within the body. These can include such precursor materials that might otherwise be considered relatively biologically inert or otherwise not effective for a particular result related to the medical condition to be treated prior to such modification.

Combinations, blends, or other preparations of any of the foregoing examples can be made and still be considered bioactive materials within the intended meaning herein. Aspects of the present invention directed toward bioactive materials can include any or all of the foregoing examples.

Stents: As used herein the term “stents” refers to devices that are used to maintain patency of a body lumen or interstitial tract. There are two categories of stents; those which are balloon expandable (e.g., stainless steel) and those which are self-expanding (e.g., nitinol). Stents are currently used in peripheral, coronary, and cerebrovascular vessels, the alimentary, hepatobiliary, and urologic systems, the liver parenchyma (e.g., porto-systemic shunts), and the spine (e.g., fusion cages). In the future, stents will be used in smaller vessels (currently minimum stent diameters are limited to about 2 millimeters). For example, they will be used in the interstitium to create conduits between the ventricles of the heart and coronary arteries, or between coronary arteries and coronary veins. In the eye, stents are being developed for the Canal of Schlem to treat glaucoma.

Substantially Simultaneously: As used herein, the term “substantially simultaneously” means that all sections of a self-expanding stent can expand closer together in time than would otherwise be allowed by the withdrawal of a retractable sheath.

### DETAILED DESCRIPTION

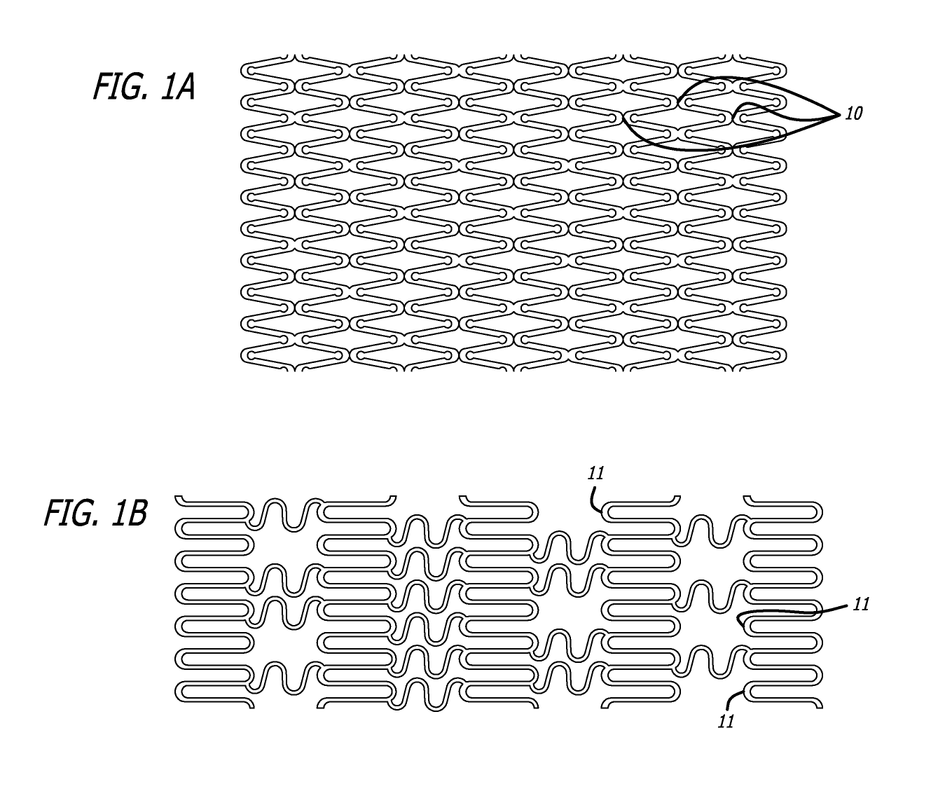

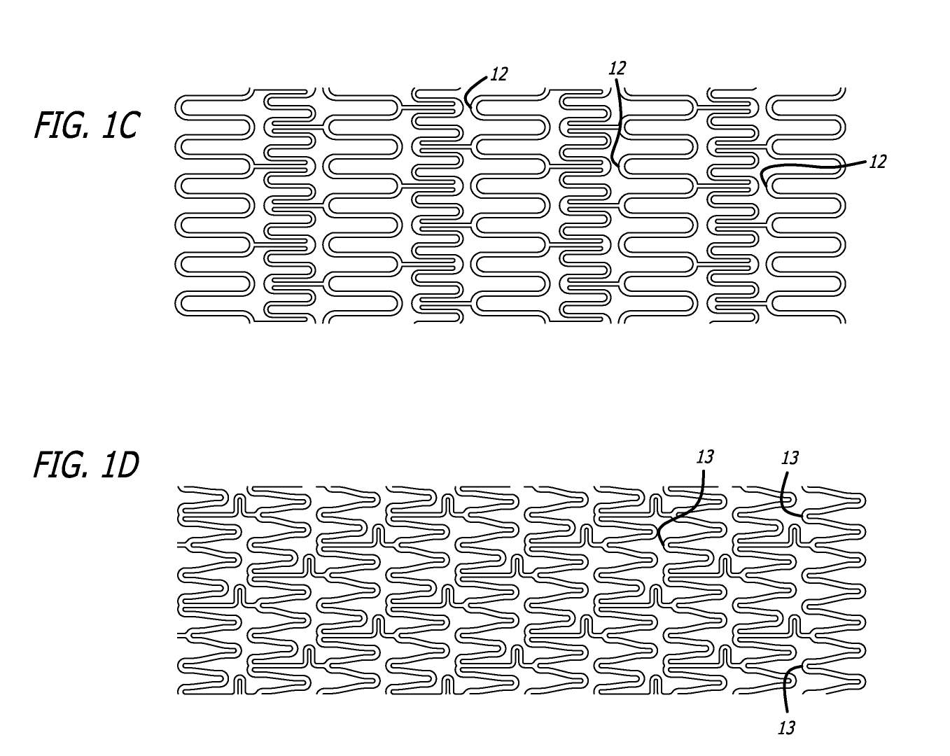

FIGS. 1A-1D depict four different exemplary stent configurations useful in accordance with the teachings of the present invention. The stents used in accordance with the present invention can have more or less undulations and/or sections than are shown in FIGS. 1A-1D but these simplified depictions are sufficient to illustrate the present invention. In these examples depicted in FIGS. 1A-1D, the stent bodies are unitary, meaning that they are fashioned from a single piece of material. For example, the stent bodies are cut to an appropriate length from appropriate material tubing. The tubing is then laser cut to form the strut pattern. In another embodiment of the present invention, the stent bodies need not be unitary but instead can consist of individual segments joined by welding, soldering, adhesive bonding, mechanical fastening, or in any other suitable way.

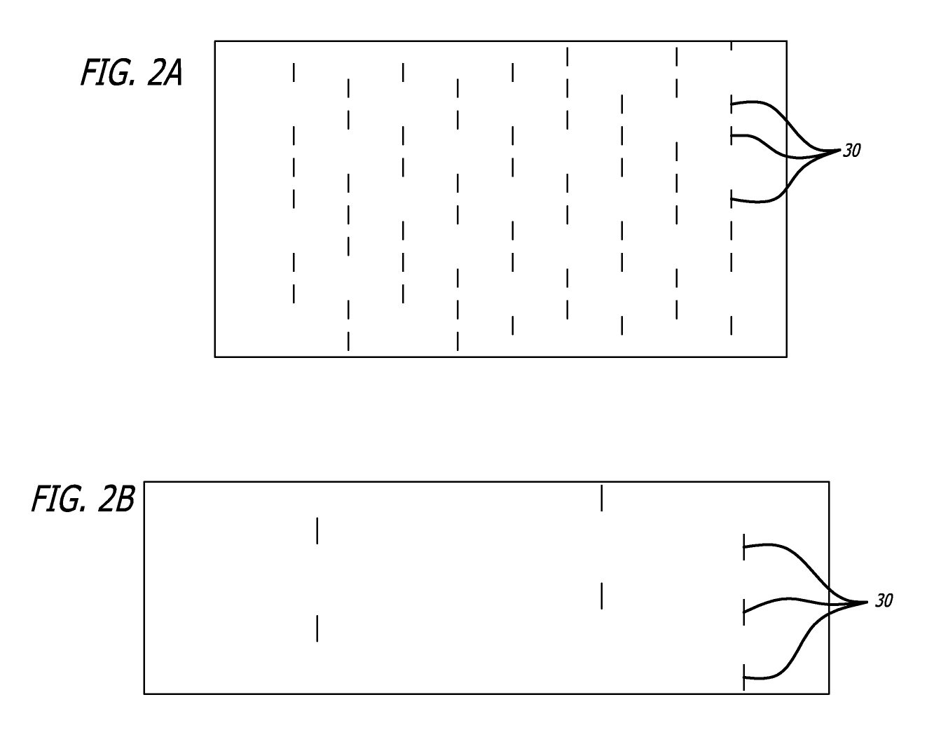

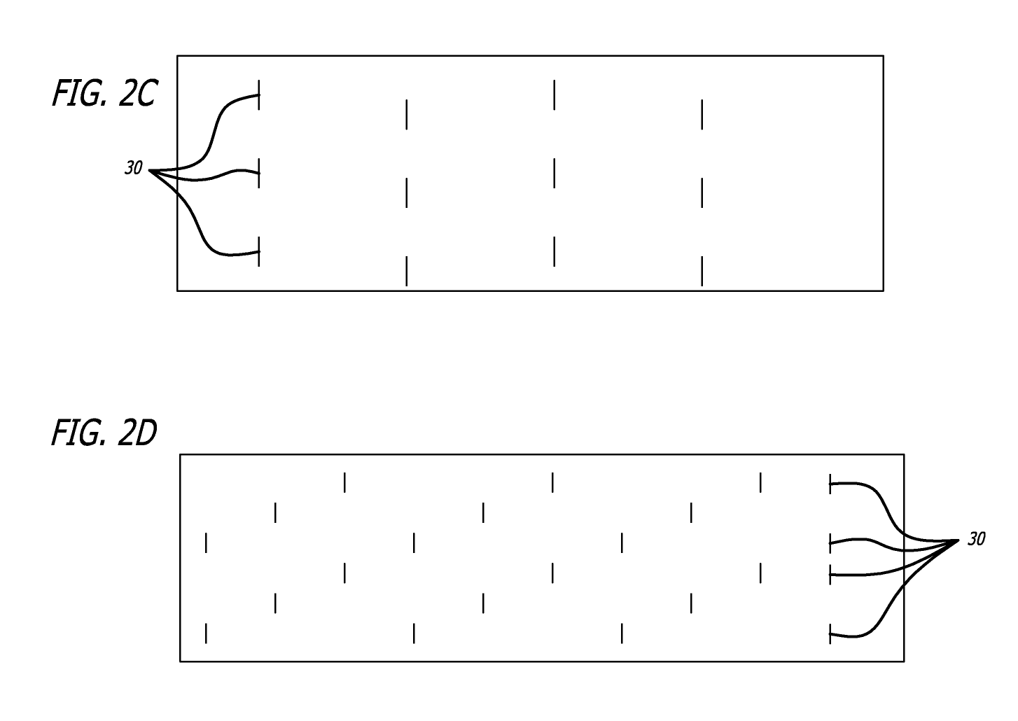

Once a stent body configuration is chosen, a device with an appropriately-sized and spaced slotted design can be manufactured (note that “slots” can include openings with or without pockets or flaps thereunder). FIGS. 2A-2D depict non-limiting and exemplary slotted designs for the stent configurations depicted in FIGS. 1A-1D respectively. As can be seen from these FIGS. 2A-2D slots 30 can be placed to accept appropriate portions of stent sections (non-limiting examples of such appropriate portions in a longtitudinal slotted design include 10 in FIG. 1A, 11 in FIG. 1B, 12 in FIGS. 1C and 13 in FIG. 1D). In one embodiment of the slotted devices of the present invention, a slot 30 can be provided for each such portion 10, 11, 12, 13 found on a stent. A slot 30 for each such portion is not required, however, and a given device must only include enough slots 30 to maintain a particular self-expanding stent in compressed form during delivery to an intended treatment site.

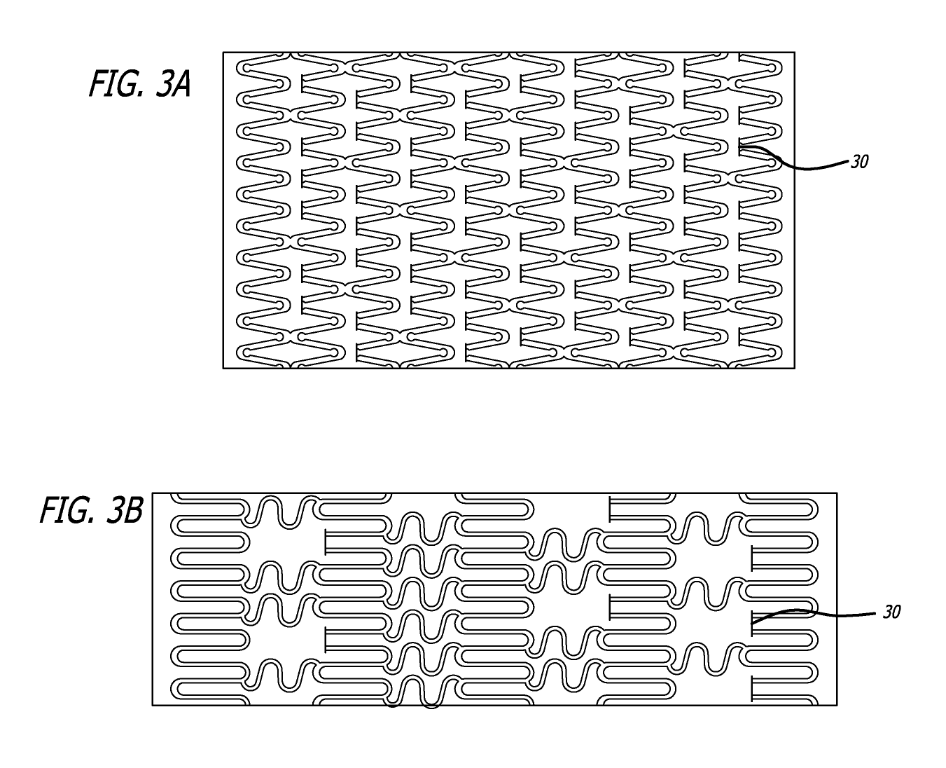

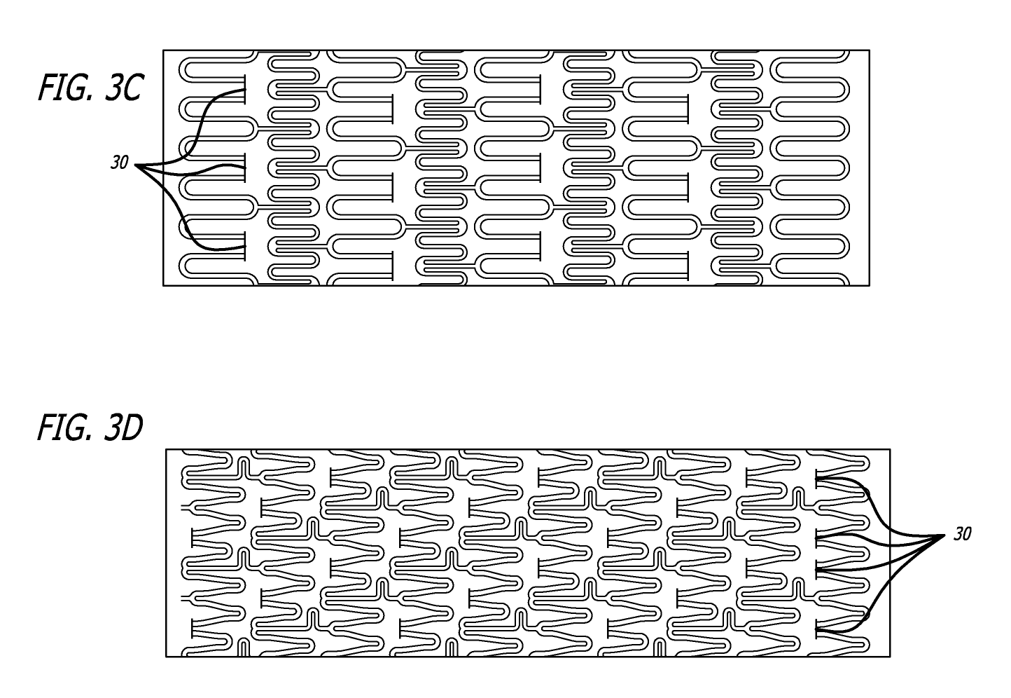

FIGS. 3A-3D depict the stent configurations depicted in FIGS. 1A-1D positioned within the slotted designs depicted in FIGS. 2A-2D respectively. As can be seen, portions 10, 11, 12, 13 are confined within the slots 30 provided on the devices of the present invention.

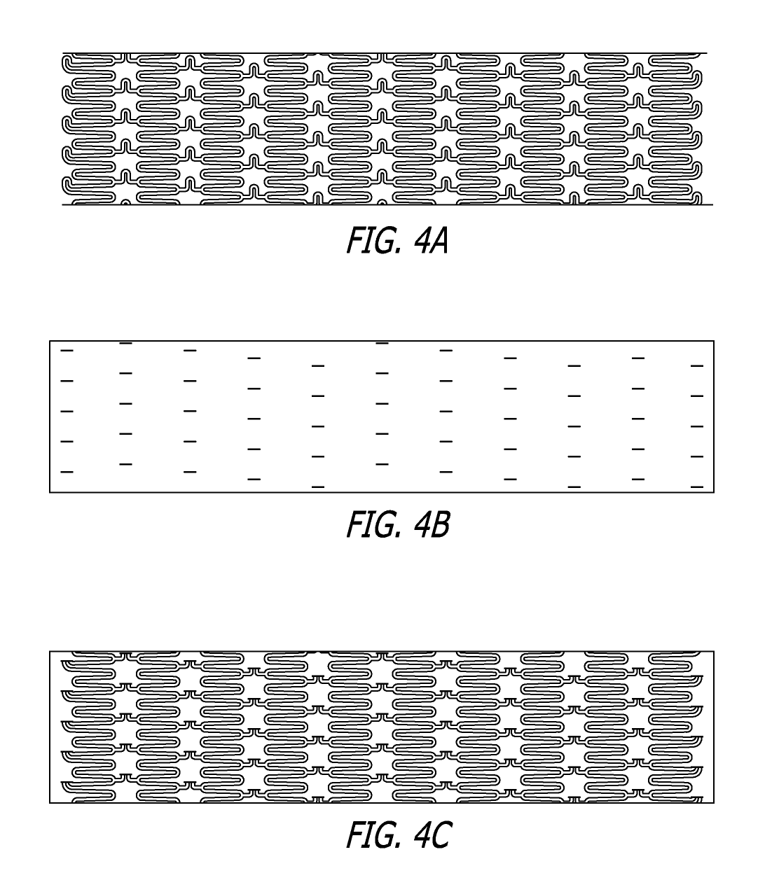

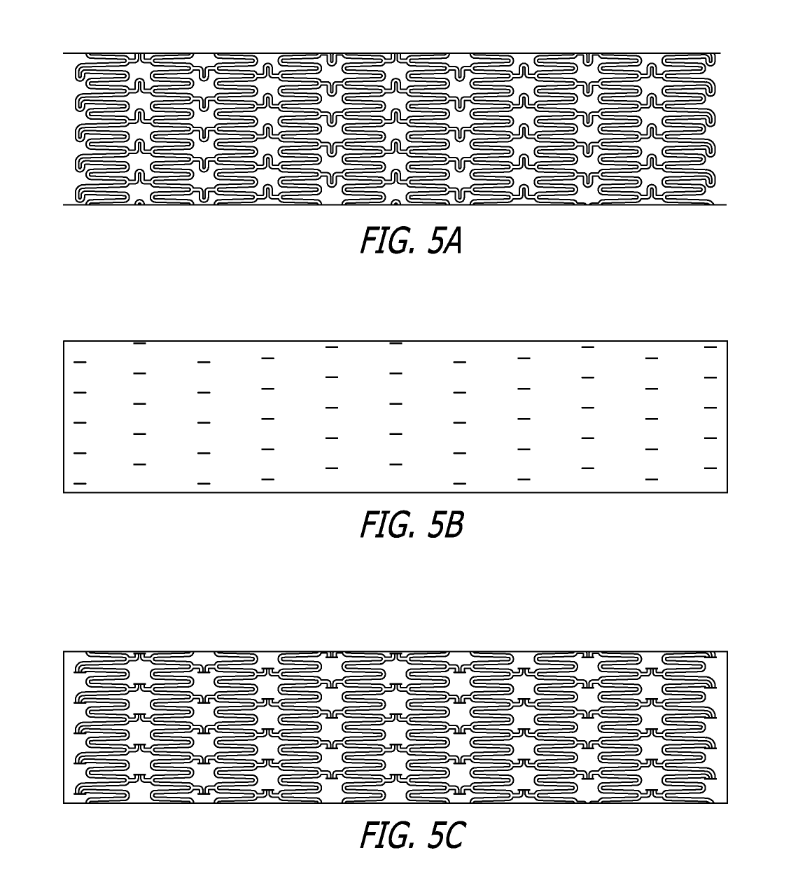

FIGS. 4A-4C and 5A-5C depict stent configurations appropriate for use with circumferential slotted designs according to the present invention (4A and 5A). Under each appropriate stent configuration is an appropriate circumferential slotted design (4B and 5B) with the stent positioned within the slotted device therebelow (4C and 5C). These depicted embodiments should make it clear that a variety of stent and slot design configurations can be made to appropriately restrain a chosen self-expanding stent.

As will be understood by one of ordinary skill in the art, the slotted devices of the present invention can be part of a delivery catheter system wherein the slotted device is attached to the distal end of a control mechanism of the delivery catheter. Once a self-expanding stent is confined in a compressed form by a slotted device of the present invention and is positioned at an intended treatment site, the controlling mechanism attached to the slotted device can move the slotted device proximally in relation to the distal end of the delivery catheter (longitudinal slot design) or can rotate the slotted device (circumferential slotted design). The controlling mechanism could also expand the slotted device (longitudinal or circumferential slotted design). Movement or expansion of the slotted device in these manners can release all sections of a self-expanding stent substantially simultaneously, thus avoiding many of the described problems associated with retractable sheaths. In contrast to a retractable sheath that must be moved proximally along the entire length of a self-expanding stent to fully deploy the stent, the slotted device of the present invention must only be moved or expanded a sufficient amount to release, for example, portions 10, 11, 111, 12, 122, 13 and 133 as depicted in FIGS. 1A-1D. Slotted designs according to the present invention can also be manufactured to include independently controlled sections such that portions of the stent can be allowed to expand independently of other portions when desired.

In addition to the described slotted devices, the present invention also includes tethering systems that can be used to maintain a self-expanding stent in a compressed form during delivery to an intended treatment site. The tethering systems, which will be hereinafter described in more detail, also reduce the occurrence of problems associated with release of self-expanding stents through withdrawal of an outer retractable sheath.

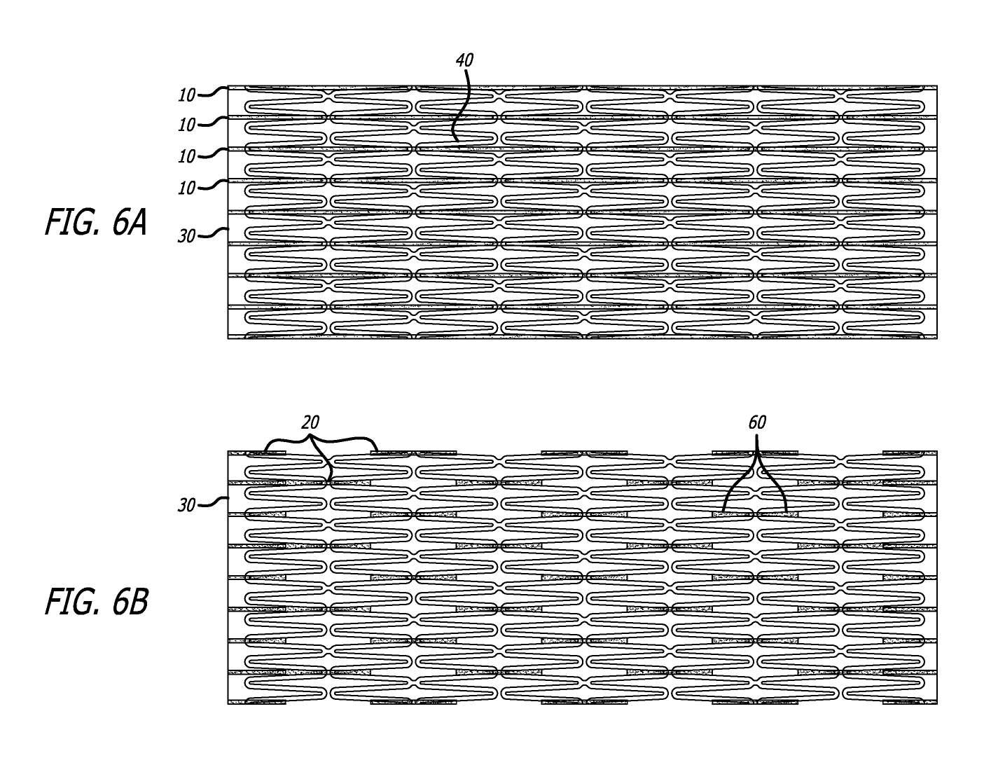

FIGS. 6A and 6B depict two alternative embodiments of longitudinal tethering systems according to the present invention. As can be seen in FIGS. 6A and 6B, the tethers are connected to a substrate at both ends. Between the ends of each tether 10, 20 and between the surface of the substrate 30 and the tethers 10, 20 is a portion of a self-expanding stent 40, 60 that is retained thereunder. The substrate can comprise, without limitation, a balloon or a sleeve thereover in which case expansion of the balloon can be used to loosen or break the tethers. The substrate need not be a balloon in all embodiments, however, and when the substrate is not a balloon, tethers or sections of tethers can be controlled (i.e. loosened or broken) by, without limitation, pull wire activation, other mechanical means, heat, current, electrical charge and combinations thereof.

While a preferred embodiment of tethering according to the present invention includes all tethers loosening or breaking substantially simultaneously, other embodiments allow for independent loosening or breaking of tethers or sections of tethers. When a balloon (or sleeve thereover) is adopted as a substrate, this can be achieved by, without limitation, using tethers of different strengths or a balloon with non-uniform (in one embodiment sequential) expansion properties. Control wires can also be used to independently control tethers or sections of tethers through a variety of means well known to those of skill in the art, including, without limitation, the previously mentioned pull wire activation, other mechanical means, heat, current, electrical charge and combinations thereof.

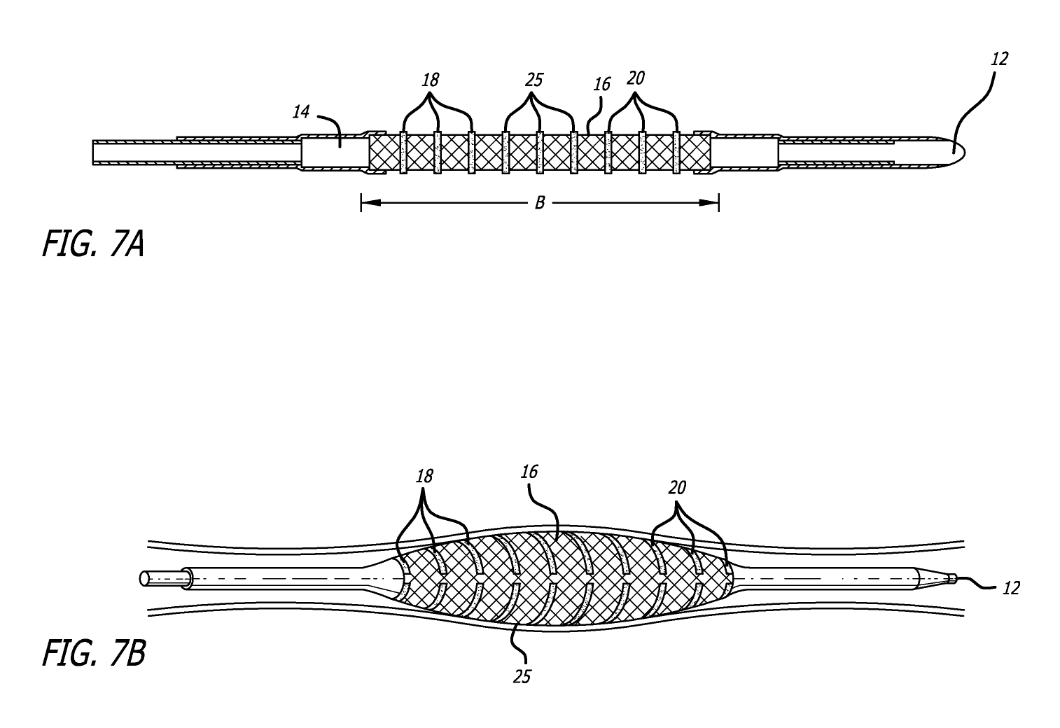

Turning to circumferential tethering and referring to FIG. 7A, in use, a balloon 14 of length B is fixed to catheter 12 using methods known to those skilled in the art. Balloon 14 is shown in its deflated and compressed form in FIG. 7A. A stent 16 is crimped about balloon 14 while tethers 18, 20, 25 maintain stent 16 in a compressed form about the balloon 14.

During deployment of the stent 16 at an intended treatment site, the balloon 14 is expanded as part of a routine angioplasty procedure. As shown in FIG. 7B, expansion of the balloon 14 causes tethers 18, 20, 25 to break (as stated, certain embodiments of tethers loosen rather than break). Once tethers 18, 20, 25 break due to balloon 14 expansion, the tethers 18, 20, 25 release the self-expanding stent 16 to deploy and stent a portion of the vessel. In one embodiment of the present invention, one point of tethers 18, 20, 25 remains attached to balloon 14 (or a sleeve thereover) and the tethers 18, 20, 25 are removed from the treatment site when the delivery catheter is removed. If required, a lubricating solution can be provided on tethers 18, 20, 25 to aid in release of stent 16 from tethers 18, 20, 25. In another embodiment, tethers 18, 20, 25 are not attached to balloon 14 (or a sleeve thereover), but instead remain attached to a portion of the stent 16. In another embodiment, tethers 18, 20, 25 are quickly biodegradable. In another embodiment, tethers 18, 20, 25 are dissolvable.

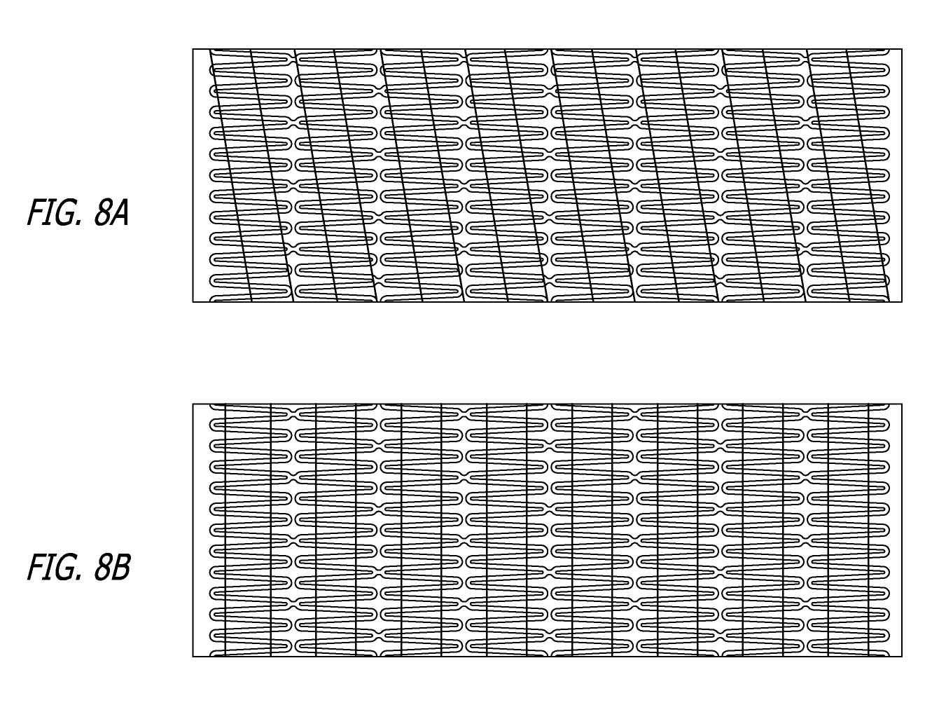

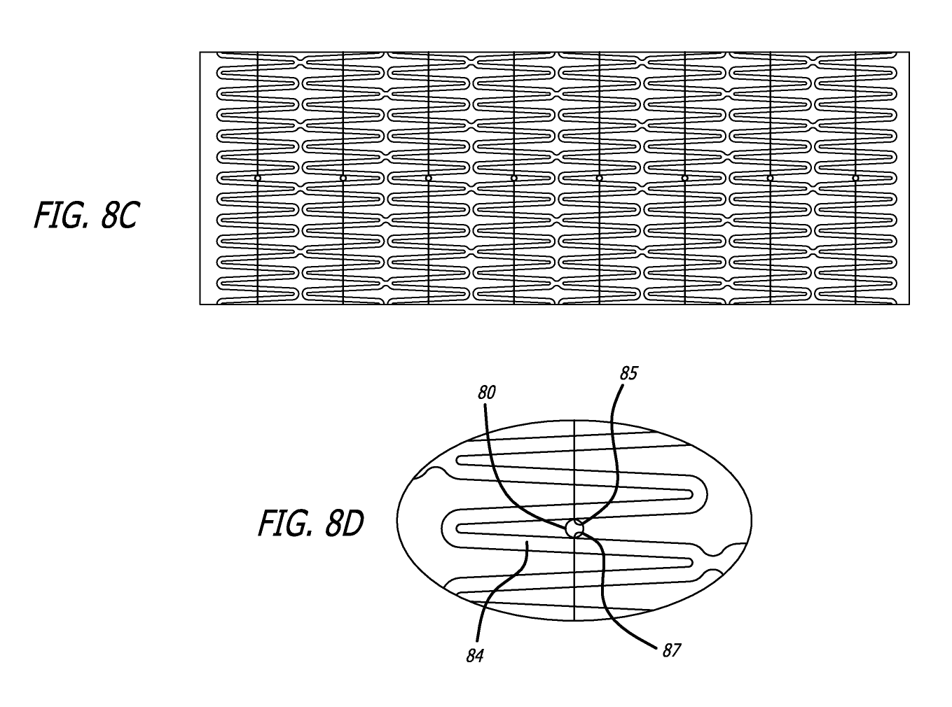

The tethers depicted in FIGS. 7A and 7B are depicted as nine tethers, each surrounding the entire circumference of a self-expanding stent. The present invention is not so limited, however. For example, a device, system or method of the present invention could use more or less than nine tethers as long as the amount of tethers used could hold the stent in a compressed form during delivery. For example, FIGS. 8A-8C depict additional embodiments of circumferential tethering. FIG. 8D shows a close up view of one example of how tethers could be attached to a sleeve over a balloon catheter and released during balloon expansion. Specifically, a small hole 80 in a sleeve overlying an inflation balloon can except both ends 85, 87 of a circumferential tether. The ends 85, 87 can be secured in place sufficiently that they will not release before balloon expansion but not so tightly that they will not release when appropriate. This appropriate degree of security can be achieved by, for example and without limitation, an adhesive found between the sleeve and inflation balloon (not shown) such that when the balloon is expanded, the ends 85, 87 of the circumferential tether will exit hole 80 and allow expansion of the self-expanding stent 84 retained thereunder.

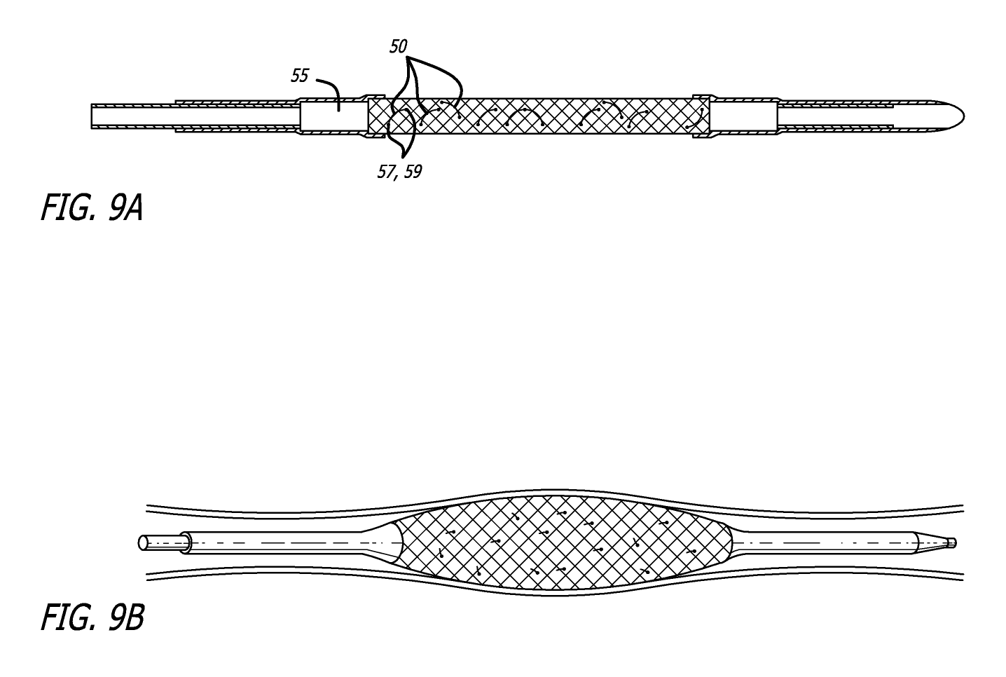

FIGS. 9A and 9B depict an additional potential embodiment of a tethering system of the present invention. In this depicted embodiment, each tether 50 is attached to an expandable balloon 55 (or a sleeve thereover) in two places, 57, 59. The attachment points 57, 59 are spaced so that the chosen tethering material is fairly taut over the material's underlying stent section. In this embodiment, the tethering materials are strong enough to maintain a self-expanding stent in a compressed form about an inflatable catheter balloon. As should be apparent to one of skill in the art, however, a weaker material can be used when more portions of the stent are tethered. Fewer portions of a self-expanding stent can be tethered when a stronger tethering material is used. Regardless of which approach is adopted, tethers 50 must break upon the application of internal pressure from the inflatable delivery catheter balloon. In the embodiment depicted in FIGS. 9A and 9B, broken tethers are removed from the treatment site with the delivery catheter.

As stated previously, in certain embodiments, tethers can loosen rather than break. In these embodiments, controls that can re-tighten loosened tethers can be employed so that expanded stents can be retracted and repositioned when necessary. For example, individual tethers or sections of tethers can be formed with an elastic material that is held taut through one or more control wires within a delivery catheter. The control wires can allow the tethers to expand and then require the tethers to re-tighten (retracting the since expanded stent) as desired.

Tethers of the present invention can be constructed from a variety of materials. Non-limiting examples of such materials include polyamides (nylons, Pebax, etc.), polyolefins (HDPE, LLDPE, etc.), polyesters (PET, Dacron, Teflon, etc.), polyimides, polyurethanes, metals, wires, and/or ribbons. In one embodiment, when breaking tether systems are employed with stronger tethering materials, the tethering material or form can be perforated to help to ensure that it breaks when appropriate.

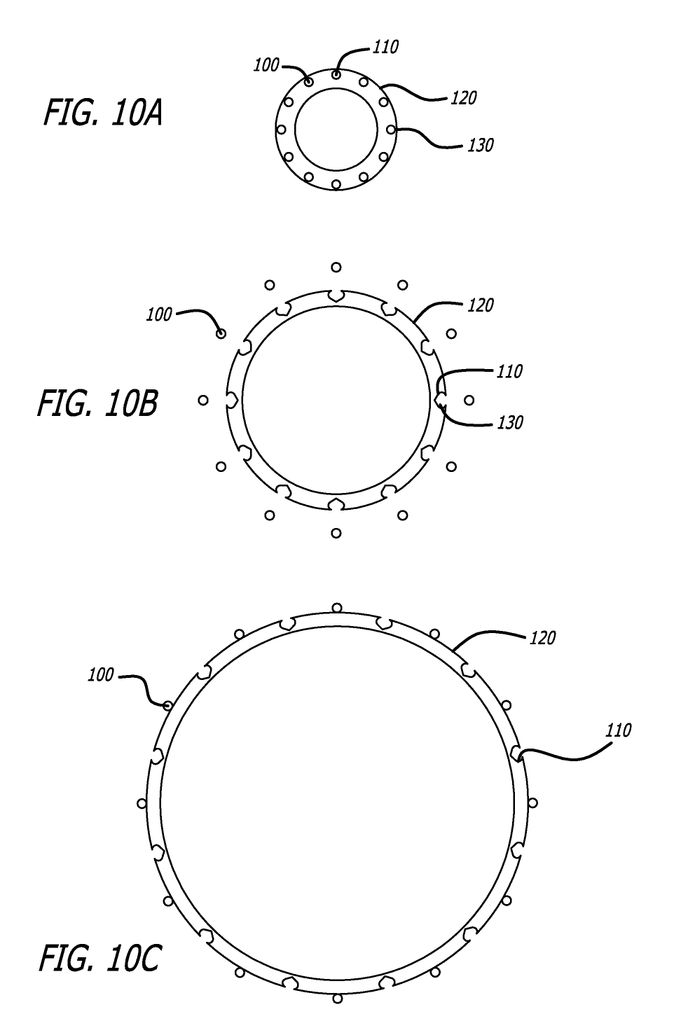

FIGS. 10A-10C depict the end view of an additional mechanism to retain self-expanding stents in a compressed form during delivery to a treatment site. In these FIG. 10, FIG. 10A depicts the ends of a self-expanding stent 100 held within channels 110 of an encapsulating flexible sheath 120. Each channel 110 has a small break 130 through which portions of the stent 100 can escape when the encapsulating flexible sheath 120 is expanded. FIG. 10B depicts the ends of the stent 100 and encapsulating flexible sheath 120 after partial expansion of encapsulating flexible sheath 120. The stent 100 has been released through the now widened openings 130 of the channels 110. FIG. 10C shows that after stent 100 has been released from channels 110, the encapsulating flexible sheath 120 can be rotated slightly and expanded further to ensure full deployment of stent 100. Rotation is beneficial to ensure that portions of stent 100 do not reenter channels 110 following deployment.

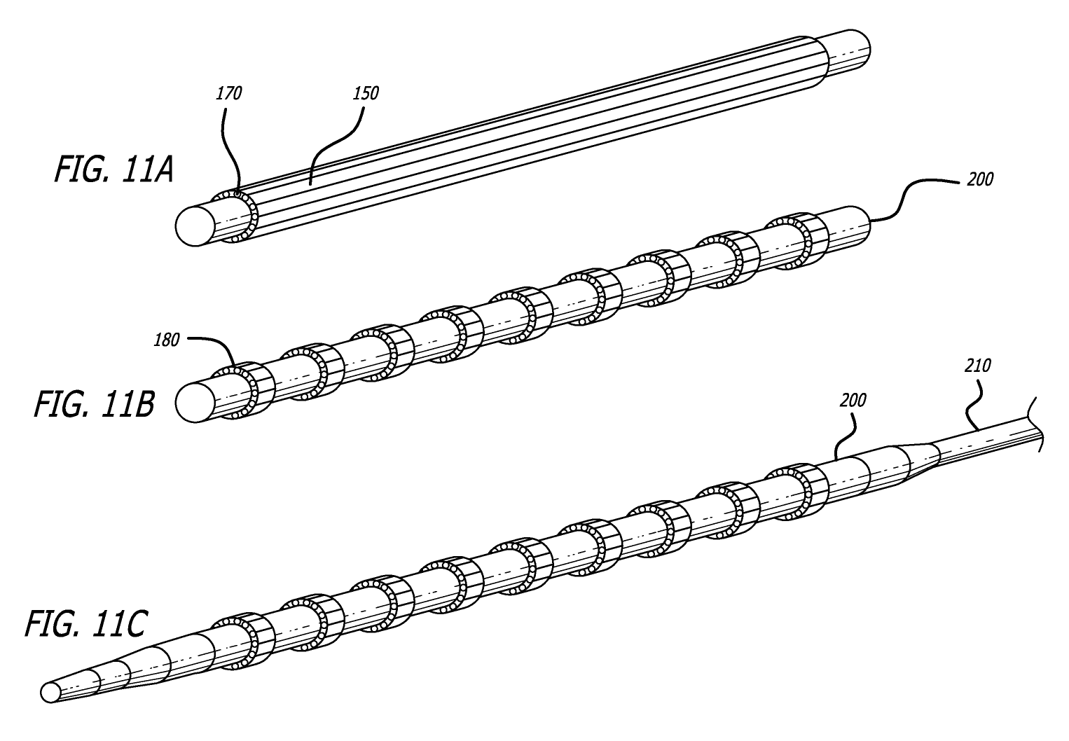

FIGS. 11A-11C provide additional views and embodiments of encapsulating flexible sheaths in accordance with the present invention. FIG. 11A depicts an encapsulating flexible sheath 150 with channels along almost its entire length. FIG. 11B depicts an alternative embodiment of a encapsulating flexible sheath 200 in accordance with the present invention that adopts channels 180 along segments of its length. In some instances, this depicted embodiment may better accommodate complex or intricate self-expanding stent designs. FIG. 11C depicts encapsulating flexible sheath 200 placed over a balloon catheter 210. Encapsulating flexible sheaths of the present invention can be manufactured from a variety of appropriate materials including, without limitation, include polyamides (nylons, Pebax®, etc.), polyolefins (HDPE, LLDPE, etc.), polyesters (PET, Dacron®, Teflon®, etc.), polyimides and polyurethanes.

Stents used in accordance with the present invention can comprise bioactive materials that can be released at the treatment site once the stent is deployed. Various methods of coating stents with bioactive materials are well-known to those of skill in the art and will not be described in detail herein. Appropriate methods for depositing bioactive materials onto the self-expanding stents of the present invention can include spray coating, dip coating, roll coating, polymer coatings, electroplated coatings, electrolessly deposited coatings, etc. Electroplating and electroless deposition are described in detail in co-pending United States Patent Application Publication Nos. 2006-0051397A1, 2006-0062820A1 and 2006-0115512A1, which are fully incorporated by reference herein.

It is to be understood that the present invention is not limited to the particular embodiments, materials, and examples described herein, as these can vary. Further, the tabs of the present invention can be made of material that is dissimilar to the material or materials that make up the other portions of the stent. It also is to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “a stent” or “a tab” is a reference to one or more stents or tabs and includes equivalents thereof known to those skilled in the art and so forth.

Unless defined otherwise, all technical terms used herein have the same meanings as commonly understood by one of ordinary skill in the art to which this invention belongs. Specific methods, devices, and materials are described, although any methods and materials similar or equivalent to those described herein can be used in the practice or testing of the present invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A stent delivery system comprising a catheter and one or more tethers wherein said one or more tethers can maintain a self-expanding stent in its compressed form when said self-expanding stent is disposed over the distal end of said catheter.

2. A stent delivery system according to claim 1 further comprising a self-expanding stent positioned over said distal end of said catheter that is maintained in a compressed form by said one or more tethers.

3. A stent delivery system according to claim 2 wherein said one or more tethers can be loosened or broken through a method selected from the group consisting of inflating a balloon; actuating a control wire; applying current; applying heat; applying electrical charge; and combinations thereof.

4. A stent delivery system according to claim 3 wherein said one or more tethers are loosened or broken substantially simultaneously.

5. A stent delivery system according to claim 3 wherein after said one or more tethers are loosened or broken at a treatment site, said tethers dissolve, degrade or are collected into a receptacle for removal from said treatment site.

6. A stent delivery system according to claim 1 wherein said catheter comprises a substrate to which said one or more tethers are attached.

7. A stent delivery system according to claim 6 wherein said substrate is an inflatable balloon or a sleeve thereover.

8. The stent delivery system according to claim 1, wherein said one or more tethers comprise a material or form selected from the group consisting of polyamides, polyolefins, polyesters, polyimides, polyurethanes, metals, wires, ribbons, and combinations thereof.

9. The stent delivery system according to claim 8, wherein said material is perforated.
